(19) United States
(12) Patent Application Publication
(10) Pub. No.: US 2022/0203002 A1
(43) Pub. Date: Jun. 30, 2022

(54) RECIRCULATION MEASUREMENT BY MEANS OF DIFFUSION EQUILIBRIUM

(71) Applicant: B. Braun Avitum AG, Melsungen (DE)

(72) Inventors: Nina Aldag, Hannover (DE); Waldemar Janik, Melsungen (DE); Silvie Krause, Melsungen (DE); Henrik Wolff, Melsungen-Adelshausen (DE)

(21) Appl. No.: 17/601,825

(22) PCT Filed: Apr. 14, 2020
(86) PCT No.: PCT/EP2020/060435

§ 371 Date: Oct. 6, 2021

(30) Foreign Application Priority Data
Apr. 17, 2019 (DE) 10 2019 110 218.9

(51) Int. Cl. A61M 1/16; G01N 21/33; G01N 21/359; G01N 21/3577

(52) U.S. Cl. CPC A61M 1/1609; A61M 1/165; A61M 1/1694; A61M 2205/3334; G01N 21/359; G01N 21/3577; A61M 2205/52; G01N 21/33

(57) ABSTRACT

An extracorporeal blood treatment machine includes a dialyzer and a sensor device downstream of the dialyzer on a dialysis fluid side. The machine is connected to a control and computing unit configured to qualitatively and quantitatively determine at least one preferably selected or selectable blood component in the used dialysis fluid. The control and computing unit is adapted to put the machine into a mode in which a dialysis fluid amount is confined within the dialyzer at least until the concentrations of the blood component on the dialysis fluid side and on the blood side of the dialyzer are in equilibrium, and thereupon to switch the machine into a mode in which the dialysis fluid flow is permitted to leave the dialyzer to feed the confined dialysis fluid amount as a dialysis fluid bolus to the sensor device for determining the blood component concentration contained in the bolus.

### CROSS-REFERENCE TO RELATED APPLICATIONS

This application is the United States national phase entry of International Application No. PCT/EP2020/060435, filed Apr. 14, 2020, and claims the benefit of German Application No. 10 2019 110 218.9, filed Apr. 17, 2019. The contents of International Application No. PCT/EP2020/060435 and German Application No. 10 2019 110 218.9 are incorporated by reference herein in their entireties.

### FIELD

The invention relates to a device for recirculation measurement in an extracorporeal blood treatment, for instance, hemodialysis, hemofiltration, and/or hemodiafiltration.

### BACKGROUND

In an extracorporeal blood treatment, for instance, a blood purification in the form of a hemodialysis, hemofiltration, or hemodiafiltration, blood is taken from a dialysis patient through an arterial vascular access, is treated in a dialyzer, and is subsequently returned to the patient via a venous vascular access. In the case of patients having a chronical disease the extracorporeal blood treatments are performed so frequently that the vein through which the blood is returned after the treatment would become inflamed and agglutinate in the long run. For this reason, those patients are supplied with a so-called shunt by surgery, said shunt constituting a cross link between the artery and the vein of the patient and being used as a permanent puncture point. Due to the shunt the vessel wall of the vein will thicken, so that it is easier to puncture and hence enables easier access for dialysis. In most cases such shunt is integrated in the arm of a patient.

Through the cross link between the artery and the vein, however, a blood exchange of venous and arterial blood also takes place, especially if, during the blood treatment, the blood flow at the blood pump is set too high and consequently the blood to be returned into the patient's shunt partially passes over to the arterial vascular access of the patient. Consequently, the venous blood already purified dilutes the unpurified, arterial blood, so that a negative impact onto the blood treatment efficiency and/or the degree of effectiveness of the blood treatment occurs. Thus, the time of treatment is increased. Such process, in which the blood already purified and returned to the patient flows from the patient's vein through the vascular access, for instance, the shunt, into the artery and from there gets again into the extracorporeal blood circulation is called recirculation and may be determined qualitatively as well as quantitatively by means of different methods. A quantitative recirculation measurement is inter alia used for monitoring the shunt state and examining the blood treatment settings, for instance, the pumping rate of the blood pump. From the state of the art, several different methods for determining the recirculation share are known. A frequently used method provides, for instance, the generation of a defined temperature bolus which is supplied in the venous blood branch, with subsequent temperature measurement in the arterial branch, so that, based on the supplied temperature difference and the measured temperature difference, the recirculation quote can be deduced mathematically. Likewise, it is known to measure, instead of the temperature, another indicator, for instance, a particular substance concentration or the conductivity, which was supplied before in the form of an (indicator) bolus. The bolus may also be supplied on the dialysis fluid side upstream of the dialyzer and a corresponding measurement from which the recirculation can be determined may be performed on the dialysis fluid side downstream of the dialyzer.

Patent document DE 197 02 441 C1, for instance, discloses a device and a method for determining the recirculation in an extracorporeal blood treatment by using a shunt, wherein an indicator parameter, for instance, a concentration bolus, is generated in the dialysis fluid circulation upstream of the dialyzer and the concentration is observed a defined time span later in the dialysis fluid circulation downstream of the dialyzer so as to draw a conclusion on the recirculation.

Also EP 2 783 715 A1 discloses a method for recirculation measurement in which a recirculation can be determined by a blood-side bolus addition and a dialysis fluid-side spectrophotometric measurement.

As a further example, patent application U.S. Pat. No. 5,588,959 A describes a device and a method for a recirculation measurement by means of temperature. Here, the blood is cooled at the venous limb section and the temperature of the blood is measured in the arterial limb section.

Further, patent application WO 96/08305 A1 discloses a method for recirculation determination in extracorporeal blood treatments in that an indicator is added in the venous blood branch and a measured value assigned to the indicator is determined by means of a detector at the arterial blood branch and a recirculation rate is calculated by means of the dilution curve.

Furthermore, methods for recirculation determination are known in which the recirculation is calculated by means of the following equation:

$$R = \frac{1 - \frac{{c}_{DO} \cdot {Q}_{D}}{{c}_{sys} \cdot {R}_{D}}}{1 - \frac{{c}_{DO} \cdot {Q}_{D}}{{c}_{sys} \cdot {K}_{D}} + \frac{{c}_{DO} \cdot {Q}_{D}}{{c}_{sys} \cdot {Q}_{B}}}$$

Here, the following applies:

| | | |
|---|---|---|
| R | recirculation rate | |
| cDO | concentration of a substance at | measured value (current) and/ |
| | the dialysate output | or measurable |
| csys | c_sys systemic concentration of | measured value/peak value |
| | a blood component which is not | |
| | affected by recirculation | |
| | (concentration in the patient) | |
| QD | dialysis fluid flow rate | known and/or adjustable |
| QB | blood flow rate in the | known and/or adjustable |
| | extracorporeal blood line system | |
| KD | theoretical clearance of the | function of (K0A, QB, QD) → |
| | dialyzer | thus known |
| K0A | dialyzer-specific | known (lookup table), i.e. |
| | | specific clearance coefficient |
| | | which is dependent on the |
| | | currently used dialyzer and |
| | | the dialysis fluid used and |
| | | which can be determined |
| | | (empirically) in advance |

This formula can be derived as follows:

The recirculation rate R is generally determined according to the following equation:

$$R = \frac{1 - \frac{{K}_{E}}{{K}_{D}}}{1 - \frac{{K}_{E}}{{K}_{D}} + \frac{{K}_{E}}{{Q}_{B}}} \qquad ( 1 )$$

with
R recirculation rate (0 . . . 1 or 0 . . . 100%)
K_{E} effective clearance
K_{D} theoretical clearance of the dialyzer
Q_{B} blood flow rate in the extracorporeal blood line system (known since adjustable at the machine)
(cf. DE 10 2013 103 221 A1, section [0123])

If the recirculation is 0, there applies K_{E}=K_{D}. The value K_{E} may accordingly be affected by recirculation.

The following relation further applies:

$${c}_{DO} \cdot {Q}_{D} = {c}_{B I} \cdot {K}_{D} = {c}_{syz} \cdot {K}_{z} \qquad ( 2 )$$

with
Q_{D} dialysis fluid flow rate (known since adjustable at the machine)
c_{DD} concentration of a substance at the dialysate outlet
c_{Bl} concentration of a blood component in the arterial branch of the blood line system
c_{sys} systemic concentration of a blood component which is not affected by recirculation (concentration in the patient).

Thus, the effective clearance may be determined by the rearranging of equation (2) as follows:

$${K}_{E} = \begin{matrix}{c}_{DO} \cdot {Q}_{D} \\ {c}_{sys}\end{matrix} \qquad ( 3 )$$

Pursuant to the equation of Michaels (Michaels. “Operating Parameters and Performance Criteria for Hemodialyzers and other Membrane—Separation Devices”. In: Trans Amer Soc Artif Intern Organs 12 (1966), 387-392.) K_{D} may be determined if the flow rates Q_{D} and Q_{E} as well as the dialyzer-specific K_{Q}A value are known:

$${K}_{D} = {Q}_{B} ⌊ \frac{exp [ \frac{{K}_{0} A}{{Q}_{B}} ( 1 - \frac{{Q}_{B}}{{Q}_{D}} ) ] - 1}{exp [ \frac{{K}_{0} A}{{Q}_{B}} ( 1 - \frac{{Q}_{B}}{{Q}_{D}} ) ] - \frac{{Q}_{B}}{{Q}_{D}}} ⌋ \qquad ( 4 )$$

The K_{Q}A value is known for the different dialyzers and may, for instance, be taken from a lookup table stored in the memory of the dialysis machine.

Inserting of the equations (3) in (1) yields:

$$R = \frac{1 - \frac{{c}_{DO} \cdot {Q}_{D}}{{c}_{sys} \cdot {K}_{D}}}{1 - \frac{{c}_{DO} \cdot {Q}_{D}}{{c}_{sys} \cdot {K}_{D}} + \frac{{c}_{DO} \cdot {Q}_{B}}{{c}_{sys} \cdot {Q}_{B}}} \qquad ( 5 )$$

Hereby, K_{D} is known from equation (4).

The above relations only apply in the case that the ultrafiltration rate is set to minimal and/or zero at the time of measurement.

In the known methods which make use of this equation (3) for recirculation measurement, first of all a blood sample of the patient is taken prior to the blood treatment for the determination of c_{sys}, and c_{sys} is measured. Subsequently, the recirculation R may be calculated during the blood treatment by means of equation (3).

The state of the art, however, always has the disadvantage that either an increased technical effort, for instance, by providing tempering devices or devices for adding a substance and/or concentration bolus, is required, or that a separate blood value determination has to take place prior to the treatment. Furthermore, the determination of c_{sys} by means of a patient's blood sample is laborious and therefore not satisfactory.

### SUMMARY

It is thus an object of the invention to overcome or at least mitigate the disadvantages from the state of the art and in particular to provide a device for recirculation measurement in an extracorporeal blood treatment, for instance, by using a shunt, which can be performed without additional technical effort, for instance, a tempering device, and/or procedural effort such as, for instance, a separate blood sample withdrawal prior to the treatment.

A basic idea of the invention consists in creating a device for the extracorporeal blood treatment which is configured to determine the systemic blood component concentration c_{sys} (concentration of a component in the blood of the patient's body) during blood treatment, and in particular exclusively with the devices basically provided at a machine for the extracorporeal blood treatment, and to then determine the recirculation R by means of the afore-mentioned, known equation (5). In other words, it shall be possible to determine c_{sys} without requiring equipment such as, for instance, a tempering device for generating a temperature bolus, or devices for injecting a substance bolus.

For this purpose, the invention makes use of a preferably optically operating sensor device at or downstream of a dialysis fluid-side dialyzer outlet, which is generally used for detecting/determining particular blood components, such as uremic toxins (e.g. urea) in the used dialysis fluid, and of a preferably dialysis machine-inherent electronic control which is, according to the invention, provided and adapted to put the dialysis machine into a mode in which dialysis fluid is confined within the dialyzer until the concentration of the blood component on the dialysis fluid side and the blood side of the dialyzer is in equilibrium, which equilibrium is no longer (or unsubstantially) changing. This means that during the confinement phase the purification of the blood in the dialyzer decreases and approaches zero, which is why finally a diffusion equilibrium exists between the blood side and the dialysis fluid side. Recirculation only plays a role here insofar as that the time span until a diffusion equilibrium has been reached changes (becomes longer).

This amount of used dialysis fluid temporarily confined in the dialyzer accordingly has a blood component concentration substantially corresponding to the patient's blood and may then be supplied like a dialysis fluid bolus to the sensor device which measures/determines the blood component concentration thereof. According to the invention, the value c_{sys} then corresponds to the peak in the sensor signal of the sensor device (directly) after the release of the dialysis fluid bolus from the dialyzer.

At this point it should be noted that the term “confined in the dialyzer” means specifically a confining of the dialyzer fluid on a dialyzer fluid membrane side of the dialyzer. Moreover, the term “confine” is to be understood as not flowing through and/or substantially not flowing through. Furthermore, in some circumstances the enclosed space also comprises parts of the dialysis fluid inlet/outlet line. For instance, if one assumed that, e.g. by activating a dialyzer bypass with a distinctly lower flow resistance (as compared to the dialyzer), flowing of the dialysis fluid through the dialyzer is interrupted and/or substantially interrupted, the tightly closing valves for such bypass circuit might be omitted. In this case, the fluid would not be really ‘confined’, but the flowing-through of the dialyzer with dialysis fluid would be reduced to almost zero.

Specifically, according to the invention there is provided an extracorporeal blood treatment machine, in particular a hemodialysis, hemofiltration, or hemodiafiltration machine (in the following also generally called dialysis machine), having a dialyzer comprising a dialysis fluid inlet for fresh dialysis fluid and a dialysis fluid outlet for used dialysis fluid, and further equipped with a filter membrane which separates a dialysis fluid membrane side, at which the dialyzer is connected to a dialyzer fluid circulation via a dialysis fluid inlet line and a dialysis fluid outlet line, from a blood membrane side at which the dialyzer is connected or connectable to an extracorporeal blood circulation. The dialysis machine according to the invention further preferably comprises a bypass line by means of which the dialysis fluid membrane side is optionally bypassable in a bypass mode so as to temporarily confine dialysis fluid present in the dialyzer. For this purpose, at least one respective (check) valve is provided at the dialysis fluid inlet line and the dialysis fluid outlet line between the bypass line and the dialyzer. Furthermore, the dialysis machine according to the invention is provided with a sensor device at or downstream of the dialysis fluid outlet of the dialyzer, said sensor device being adapted to metrologically, especially optically, determine blood components passing through the filter membrane, especially uremic toxins (e.g. urea, creatinine, uric acid, potassium, etc.) in the used dialysis fluid draining from the dialyzer. At this point it should be noted generally that the sensor measures a physical or chemical quantity which may be proportional to the concentration of a particular substance. Actual and/or directly measured concentration values are not required by the proposed method for recirculation measurement. The dialysis machine according to the invention further comprises a control and computing unit for controlling the dialysis machine, preferably with a memory unit. The dialysis machine is preferably further provided or adapted to be equipped with a data set stored or storable on the memory unit or a comparable separate storage medium, said data set comprising, at least for the currently connected dialyzer, a blood flow value in the extracorporeal blood circulation and a corresponding, preferably analytically determined, time value, wherein the blood flow value and the time value and/or the identity and/or the properties of the connected dialyzer might, of course, also be input manually, scanned, or be fed in some other manner. Within this period determined by the time value, with an adjusted blood flow value, assuming a maximally possible recirculation value (e.g. 20 percent), a concentration equilibrium of at least one selected or selectable blood component between blood in the extracorporeal blood circulation and dialysis fluid confined in the dialyzer is concluded exclusively due to diffusion. The dialysis machine further comprises a calculation model (stored on the memory unit) by means of which the control and calculating unit calculates an actual recirculation value, for instance, in consideration of a concentration of the at least one selected or selectable blood component in the blood of the patient's (body) or an absorbance (because concentration is equivalent to absorbance), preferably at the beginning of a treatment cycle by means of the dialysis machine. For this purpose, for determining the at least one selected or selectable blood component in the blood of the patient's (body), the control and computing unit switches the dialysis machine into the bypass mode for the duration of the time value indicated in the data set or input manually (including scan), and operates the extracorporeal blood circulation, preferably simultaneously, at the blood flow value indicated, and metrologically determines, directly after termination of the bypass mode, by means of the sensor device a concentration bolus and/or a measurement parameter representing same which was produced by the bypass mode, in the used dialysis fluid draining from the dialyzer.

In other words, for the determination of an actual recirculation during a blood treatment first of all the dialysis machine is operated temporarily in a bypass mode. In this bypass mode the dialysis fluid side in the dialyzer is not supplied with fresh dialysis fluid and/or the filter membrane on the dialysis fluid side is not passed by fresh dialysis fluid. Rather, the dialysis fluid present on the dialysis fluid membrane side is kept there, preferably by using valves arranged in the dialysis fluid inlet line and the dialysis fluid outlet line. Instead of valves, other elements suited for the blocking of fluid, for instance, pumps are, of course, also conceivable. While the dialysis fluid present on the dialysis fluid membrane side is now kept there, the blood flow in the extracorporeal blood circulation is operated at a fixed blood flow rate. The blood side of the dialyzer is thus still supplied with arterial blood and/or blood to be purified, and/or the filter membrane is still passed at the blood side by arterial blood and/or blood to be purified. Consequently, as is usual in dialysis treatments, due to diffusion a transfer of substances (blood components) solved in the blood takes place through the filter membrane into the dialysis fluid, namely until no more concentration gradient exists between the blood present on the blood side and the dialysis fluid present on the dialysis fluid side, i.e. in other words, a diffusion equilibrium has been reached. Since the blood continues flowing on the blood membrane side of the dialyzer while the dialysis fluid on the dialysis fluid membrane side of the dialyzer is stationary, blood components will enrich in the stationary dialysis fluid as long as they have reached at least substantially the same concentration in the stationary dialysis fluid as in the blood that continues flowing through the dialyzer. In other words, at the time at which the diffusion equilibrium of a blood component between the stationary dialysis fluid and the flowing blood has been (substantially) reached, the concentration of the blood component dissolved in the stationary dialysis fluid corresponds at least substantially to the concentration of the blood component dissolved in the blood of the patient's body. At this point it should be noted that the parameter “concentration” may also be represented by another parameter associated therewith, such as, for instance, an absorbance or a measurable electrical conductivity. For calculating the actual recirculation, with the reaching of the diffusion equilibrium, the concentration of a blood component present in the stationary dialysis fluid (and measurable/determinable directly or indirectly) may be taken as a substitute of the concentration c_{sys} of this blood component which is present in the blood of the patient's body. In order to compensate for a possible exceeding of the linear sensor measurement range, a compensation factor k may be used additionally. K may be determined by a function which describes the characteristic line of the sensor analytically.

The time at which a diffusion equilibrium has been reached depends, apart from the specific blood component, for instance, a particular uremic toxin, possibly also on the degree of effectiveness of the blood treatment, i.e. on the actually existing recirculation. If the recirculation is only less relevant or not existing at all, the diffusion equilibrium will possibly be reached more quickly than in the case of a high recirculation since recirculated blood results in a dilution of the arterial blood to be purified and thus impairs the efficiency of blood purification. The duration until the diffusion equilibrium between the blood side and the dialysis fluid side has been reached is, however, significantly dependent on the blood flow adjusted and on the size of the dialyzer. The larger the blood flow and the smaller the dialyzer, the quicker a diffusive equilibrium is reached.

The intensity of the recirculation, however, has an influence on the duration until the arterial blood concentration cBl corresponds to the systemic concentration cSYS. In the case of a non-existing recirculation cBl almost corresponds to cSYS. Therefore, it has to be assumed that this process takes less time than the above-mentioned generation of the diffusion equilibrium.

In order to ensure that the duration of the bypass mode also really corresponds at least to the duration until the diffusion equilibrium of a particular blood component in the dialyzer has occurred, the duration of the bypass mode must always be set to the maximum duration of the occurrence of the diffusion equilibrium. This is supplied to the control and computing unit in advance, either by retrieving the information from a data base/a data set, or by input by a user, and may be dependent on the patient, his/her shunt state, and/or further specific experience values, for instance, empirical determination in the laboratory or evaluation of past blood treatments.

The duration of the bypass mode and/or the duration to be expected until the diffusion equilibrium has occurred in the dialyzer further depend on the type of dialyzer used, for instance, on the condition (new or reused), and/or on the (available) filter face, and/or on the (dialysate-side) filling volume, and on the flow rate at which the blood to be purified is guided through the dialyzer. Therefore, a data set is available for the control and computing unit from which the target duration of the bypass mode and the target blood flow rate to be set can be determined in consideration of the maximally possible recirculation and the properties of the specifically used/connected dialyzer. Alternatively, it is also conceivable that the target duration of the bypass mode is also determined as a function of a predetermined blood flow rate.

Once the target duration of the bypass mode has been reached and consequently a diffusion equilibrium has occurred in the dialyzer for a particular blood component between the blood side and the dialyzer fluid side in the dialyzer, the bypass mode is terminated and the valves in the dialysis fluid inlet line and the dialysis fluid outlet line are thus opened again. By means of the sensor device, which is preferably arranged downstream of the valve in the dialysis fluid outlet line, it is now possible to determine, in the dialysis fluid flowing through the dialysis fluid outlet line, the concentration of the particular blood component which corresponds to the concentration of the particular blood component in the blood of the patient's body.

Advantageously, it is thus possible to determine an actually existing recirculation without the concentration of a blood component having to be determined before in the blood of a patient's body by means of a separate blood withdrawal on the patient, and consequently a fully automated, time-saving, and safe blood treatment method may be enabled.

Further preferred, the memory unit is steadily integrated in the dialysis machine. Advantageously, no external, additional storage medium, for instance, a USB stick, needs to be connected prior to the treatment.

### BRIEF DESCRIPTION OF THE DRAWING FIGURES

The invention will be described in detail in the following by means of a preferred embodiment with reference to the enclosed drawing figures, of which:

FIG. 1 a first embodiment of an extracorporeal blood treatment machine according to the invention;

FIG. 2 a second embodiment of an extracorporeal blood treatment machine according to the invention;

FIG. 3 a third embodiment of an extracorporeal blood treatment machine according to the invention;

FIG. 4 a chronological sequence of the measurements at a sensor device of a dialysis machine where the sensor device is arranged directly behind or before the valve of the dialysis fluid outlet line.

The Figures are merely of schematic nature and serve exclusively for the understanding of the invention. The same elements are designated with the same reference numbers.

### DETAILED DESCRIPTION

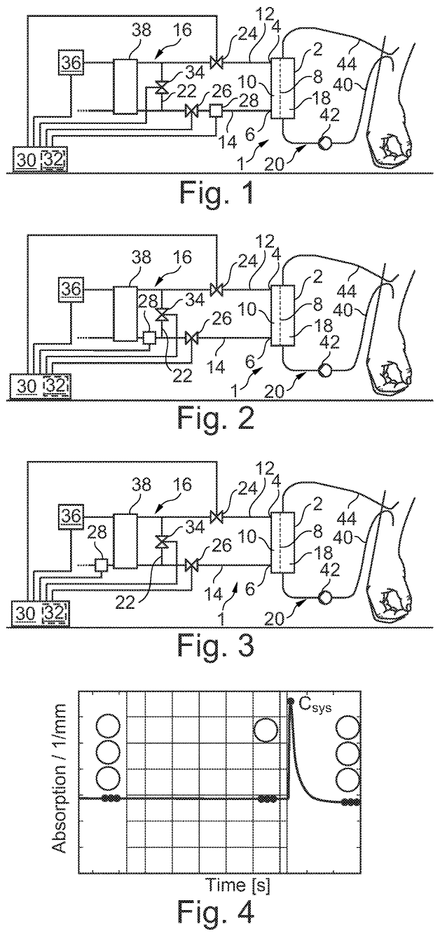

FIG. 1 illustrates a first embodiment of an extracorporeal blood treatment machine (dialysis machine) 1 according to the invention, having a dialyzer 2 comprising a dialysis fluid inlet 4 and a dialysis fluid outlet 6. The dialyzer 2 is further equipped with a filter membrane 8 separating a dialysis fluid membrane side 10, which is in fluid connection with a dialysis fluid inlet line 12 and a dialysis fluid outlet line 14 of a dialysis fluid circulation 16, from a blood membrane side 18. The dialysis fluid inlet line 12 and the dialysis fluid outlet line 14 are connected to the dialysis fluid inlet 4 and the dialysis fluid outlet 6 of the dialyzer 2. The blood membrane side 18 is in fluid connection with an extracorporeal blood circulation 20. In the dialysis fluid circulation 16, a bypass line 22 is further provided, by means of which the dialysis fluid membrane side 10 of the dialyzer 2 may be bypassed, i.e. be circumvented fluidically. In the dialysis fluid inlet line 12 and the dialysis fluid outlet line 14 a respective valve 24, 26 is provided by means of which the dialysis fluid inlet line 12 and the dialysis fluid outlet line 14 may be opened and/or closed and thus either release the flow path for fresh dialysis fluid with opened valves 24, 26 through the dialysis fluid inlet line 12, the dialysis fluid membrane side 10 of the dialyzer 2, and the dialysis fluid outlet line 14, or close the flow path by closing at least the valve 24, ideally both valves 24, 26.

In the first embodiment of the dialysis machine 1 according to the invention a sensor device 28 is positioned downstream of the dialysis fluid outlet 6 and upstream of the valve 26. It is, for instance, configured as an optical sensor device and performs, in the dialysis fluid flowing past, measurements by means of UV/VIS spectroscopy as a function of the absorption range of the specific blood component to be measured at the wavelength absorbable by this blood component, so as to enable a concentration determination of this blood component or a corresponding parameter determination. Concentration/parameter determinations by absorption measurement are generally known and will therefore not be explained here in detail. Apart from optical measurement methods, alternative measurement methods, for instance, by conductivity determination, are also conceivable. The measured values generated at the sensor device 28 are transmitted to and evaluated by a control and computing unit 30.

The control and computing unit 30 is in mutual information exchange at least with the valves 24, 26 and the sensor device 28, i.e. it receives and transmits information from the valves 24, 26 and the sensor device 28 and/or to the valves 24, 26 and the sensor device 28. Furthermore, the control and computing unit 30 is connected to or equipped with a memory unit 32. At least one data set by which the dialysis machine 1 can be controlled as a function of the predetermined and/or received information is stored and/or storable on this memory unit 32.

The dialysis machine 1 further comprises a further valve 34 in the bypass line 22 which is also in information exchange contact with the control and computing unit 30 and may be controlled, i.e. opened and closed, by it. The valve 34 is provided to release or to block the flow path for fresh dialysis fluid via the bypass line 22. The fresh dialysis fluid is obtained from a dialysis fluid providing unit/dialysis fluid source 36. A balancing chamber 38 which balances fresh dialyzing fluid flowing into the dialysis fluid inlet circulation 16 and (used) dialysis fluid flowing out thereof is positioned downstream of the dialysis fluid source 36. The balancing chamber 36 is arranged fluidically such that it is positioned between the dialysis fluid source 36 and a mouth position of the bypass line 22 in the dialysis fluid inlet line 12, and between a mouth position of the bypass line 22 into the dialysis fluid outlet line 14 and a drain.

On the blood membrane side 18 of the dialyzer 2 the extracorporeal blood circulation 20 is connected to the dialyzer 2. The blood circulation 20 comprises at least one arterial blood line 40 which connects a patient's arterial access to a blood-side dialyzer input and at which a blood pump 42 is arranged, and a venous blood line 44 which connects a blood-side dialyzer output to a patient's venous access. The blood pump 42 is also in (mutual) information exchange contact with the control and computing unit 30 and is controlled by it.

FIG. 2 illustrates a second embodiment of a dialysis machine 1 according to the invention, which differs from the dialysis machine 1 of the first embodiment merely in that the sensor device 28 is not arranged upstream of the valve 26, but is positioned fluidically between the valve 26 and the balancing chamber 38, especially between the mouth position of the bypass line 22 into the dialysis fluid outlet line 14 and the balancing chamber 38.

FIG. 3 illustrates a third embodiment of a dialysis machine 1 according to the invention, which differs from the dialysis machine 1 of the first embodiment merely in that the sensor device 28 is not arranged upstream of the valve 26, but is positioned downstream of the balancing chamber 38.

In operation of the dialysis machine 1 (all embodiments), a patient is first connected to the extracorporeal blood circulation 20. Subsequently, the control and computing unit 30 retrieves a target blood flow value and a time value for the duration of a bypass mode from the data set which is stored on the memory unit 32. Patient-dependent minimal and maximal upper limits for the target blood flow value may be taken into account in the data set. The time value for the duration of the bypass mode is preferably chosen/determined such that, during the bypass mode, an (approximate) diffusion equilibrium is reached for the blood component to be measured in the dialyzer 2 even under the worst circumstance, namely a maximally possible recirculation or a recirculation to be expected maximally (e.g. recirculation of 20 percent to 30 percent). Here, the rule applies that the lower the blood flow value and the larger the dialyzer, the longer it takes until the (approximate) diffusion equilibrium has been reached. Depending on the specific blood component to be measured/to be determined, which is chosen optionally prior to the treatment from a plurality of possible blood components, the output time value may additionally be larger or smaller.

After the control and computing unit 30 has retrieved the target time value and the target blood flow value and/or they were input manually, it sets the blood pump 42 to the target blood flow value. Once the target blood flow value has been reached and a predetermined value for the dialysis fluid flow through the dialysis fluid circulation 16 has also been reached, the dialysis machine 1 is switched to the bypass mode by the control and computing unit 30. This means that, for the duration of the bypass mode, the valves 24, 26 are closed, i.e. the dialysis fluid present between the valves 24, 26 is confined, and the valve 34 in the bypass line 22 is opened, so that the flow path of the fresh dialysis fluid leads from the dialysis fluid source 36 via the bypass line 22 to the drain. For the duration of the bypass mode, the blood pump 42 is now operated at the rate set while the dialysis fluid on the dialysis fluid membrane side 10 is stationary. Consequently, the blood component to be measured/determined passes from the blood flowing through the blood membrane side 18 of the dialyzer 2 via the filter membrane 8 into the dialysis fluid which is stationary on the dialysis fluid membrane side 10 of the dialyzer 2. Thus, the blood component to be measured/determined will enrich in the stationary dialysis fluid until a diffusion equilibrium has been (substantially) reached on the blood membrane side 18 and the dialysis fluid membrane side 10. Since the blood in the extracorporeal blood circulation 20 flows on continuously, the diffusion equilibrium concentration of the blood component to be measured/determined corresponds at that time substantially to the available concentration of the blood component to be measured/determined in the blood of the extracorporeal blood circulation 20, which corresponds at that time in turn to the available concentration of the blood component to be measured in the blood of the patient's body. Consequently, the diffusion equilibrium concentration of the blood component to be measured/determined in the stationary dialysis fluid corresponds to the concentration of the blood component to be measured in the blood of the patient's body.

Once the diffusion equilibrium has been (almost) reached and/or once the predetermined time value for the bypass mode, within which the diffusion equilibrium is deemed to have been reached, has been reached, the control and computing unit 30 ends the bypass mode. Consequently, it closes the valve 34 in the bypass line 22 and opens at the same time the valves 24, 26 in the dialysis fluid inlet line 12 and the dialysis fluid outlet line 14, so that fresh dialysis fluid flows from the dialysis fluid source 36 again through the dialysis fluid membrane side 10 of the dialyzer 2. The dialysis fluid that had been stationary before on the dialysis fluid membrane side 10 consequently flows through the dialysis fluid outlet line 14 in the direction of the drain, passing the sensor device 28. The sensor device 28 measures, as a consequence of the enrichment of the blood component to be measured, a peak (light absorption peak in place of the concentration of this blood component) whose maximum may be evaluated as a diffusion equilibrium concentration of the blood component. In conclusion, after the measurement of the peak maximum the concentration present in the blood of the patient's body of the blood component to be measured/determined is thus known, and in the subsequent and/or continuing blood treatment the actual recirculation may be calculated in a known way.

FIG. 4 illustrates the chronological sequence of the measurement (absorption value of the blood component to be measured) at a sensor device 28 of a dialysis machine 1 in which the sensor device 28 is arranged directly behind the valve 26 of the dialysis fluid outlet line 14 and before or behind the mouth position of the bypass line 22 in the dialysis fluid outlet line 14 (the temporary offset of the peak with a displacement of the sensor device 28 in downstream direction is unconsidered as being negligible in FIG. 4 for the sake of convenience). The checkered area is the duration in which the bypass mode is active, i.e. no dialysis fluid flowing from the dialysis fluid membrane side 10 flows past. Before and during the bypass mode the sensor device 28 consequently measures in the dialysis fluid a constant parameter for the concentration for a particular blood component since the dialysis fluid and/or the dialysis fluid fraction in which the blood component to be measured enriches due to diffusion is confined between the valves 24, 26 and fresh dialysis fluid from the dialysis fluid source 36 does not flow around the sensor device. The concentration thus measured may be considered as the concentration of the blood component to be measured at the dialyzer output under the normal, known treatment/operating conditions (c_{do}).

After the termination of the bypass mode the sensor device measures a distinct peak in the concentration of the blood component to be measured since, after the opening of the valve 26, the previously confined dialysis fluid fraction now flows past the sensor device 28. It has to be noted that, for the case in which the sensor device 28 is arranged directly behind the valve 26, the peak maximum corresponds almost to the diffusion equilibrium concentration. If there is a larger distance between the valve 26 and the sensor device 28, the peak maximum decreases due to diffusion, so that appropriate mathematical correction measures are used for determining the diffusion equilibrium concentration. The diffusion equilibrium concentration measured/determined of the blood component to be measured corresponds to the systemic blood component concentration c_{sys}.

Once the concentration bolus of the blood component to be measured/determined in the dialysis fluid has passed the sensor device 28 completely, the measured/determined concentration of the blood component again corresponds to c_{do}.

With the known equation (5) according to the foregoing description the recirculation R may now be calculated.

1. An extracorporeal blood treatment machine comprising a dialyzer and a sensor device that is downstream of the dialyzer on a dialysis fluid side and is electrically connected to a control and computing unit, which is provided and configured to both qualitatively and quantitatively determine, based on measurement signals of the sensor device, a blood component in a used dialysis fluid, the control and computing unit further configured to:
put the extracorporeal blood treatment machine into a first mode in which a dialysis fluid amount is confined within the dialyzer at least until a concentration of the blood component on the dialysis fluid side is in equilibrium with a concentration of the blood component on a blood side of the dialyzer, which equilibrium is no longer changing or is only still changing to an unsubstantial degree, wherein a time period within which the equilibrium is established is determined by a time value, and thereupon
switch the blood treatment machine into a second mode in which a dialysis fluid flow is permitted to leave the dialyzer in order to feed a previously confined dialysis fluid amount as a dialysis fluid bolus to the sensor device for determining a concentration of the blood component contained in the dialysis fluid bolus, and
calculate a current recirculation value from said concentration of the blood component contained in the dialysis fluid bolus and from a number of further machine and/or adjustment parameters.

2. The extracorporeal blood treatment machine according to claim 1, wherein a concentration of said blood component in the used dialysis fluid corresponds to a maximum peak in a sensor signal of the sensor device directly after a release of the dialysis fluid bolus from the dialyzer.

3. The extracorporeal blood treatment machine according to claim 1, wherein a calculation of the current recirculation value takes place using a calculation model defined by the following formula:
$$R = \frac{1 - \frac{{c}_{DO} \cdot {Q}_{D}}{{c}_{sys} \cdot {K}_{D}}}{1 - \frac{{c}_{DO} \cdot {Q}_{D}}{{c}_{sys} \cdot {K}_{D}} + \frac{{c}_{DO} \cdot {Q}_{D}}{{c}_{sys} \cdot {Q}_{B}}}$$
with

| | |
|---|---|
| R | recirculation rate |
| cDO | concentration of a substance at the dialysate output |
| csys | c_sys systemic concentration of a blood component which is |
| | not affected by recirculation (concentration in the patient) |
| QD | dialysis fluid flow rate |
| QB | blood flow rate in the extracorporeal blood line system |
| KD | theoretical clearance of the dialyzer |
| K0A | dialyzer-specific. |

4. The extracorporeal blood treatment machine according to claim 3, wherein:
the dialyzer comprises a dialysis fluid inlet for fresh dialysis fluid, a dialysis fluid outlet for used dialysis fluid, and a filter membrane which separates a dialysis fluid membrane side, at which the dialyzer is connected to a dialysis fluid circulation via a dialysis fluid inlet line and a dialysis fluid outlet line, from a blood membrane side at which the dialyzer is connected or connectable to an extracorporeal blood circulation;
a bypass line is provided by which the dialysis fluid membrane side is bypassable in a bypass mode so as to temporarily confine dialysis fluid present in the dialyzer, for which purpose at least one respective check valve is provided at the dialysis fluid inlet line and the dialysis fluid outlet line between the bypass line and the dialyzer; and
the control and computing unit is configured or adapted to be equipped with a memory unit,
wherein the extracorporeal blood treatment machine further comprises:
a data set stored or storable on the memory unit and indicating a number of blood flow values suited for different parameters of the blood treatment machine in the extracorporeal blood circulation and corresponding time values within which, in the dialyzer, with an appropriately adjusted blood flow value, assuming a maximally possible recirculation value, a concentration equilibrium of at least one selected or selectable blood component between blood in the extracorporeal blood circulation and dialysis fluid confined in the dialyzer is completed exclusively due to diffusion; and
the calculation model stored on the memory unit, by which the control and computing unit, taking into account a concentration of the blood component, calculates an actual recirculation value, for which purpose the control and computing unit, for determination of the blood component in the blood of the patient, switches the extracorporeal treatment machine into the bypass mode for a duration of the time value and operates the extracorporeal blood circulation at a blood flow value indicated and, directly after termination of the bypass mode, metrologically determines, by the sensor device, a concentration bolus produced by the bypass mode in the used dialysis fluid draining from the dialyzer.

5. The extracorporeal blood treatment machine according to claim 4, wherein the memory unit is steadily integrated in the extracorporeal blood treatment machine.

6. A method for monitoring a recirculation rate in an extracorporeal blood treatment by using the extracorporeal blood treatment machine in accordance with claim 1, comprising the following steps:
determining a time value as a function of an adjusted blood flow value within which a concentration equilibrium of a previously selected blood component occurs:
operating the extracorporeal blood treatment machine in the bypass mode in which, by maintaining the blood flow value, a dialysis fluid is confined within a dialyzer for the determined time value,
switching to a release mode in which the dialysis fluid is released in a direction of an outlet of the dialyzer;
capturing a measurement peak in a scope of a sensory measurement of a blood component-equivalent measurement parameter in the dialysis fluid after switching to the release mode; and
calculating the recirculation rate by a calculation model.

7. The method for monitoring a recirculation rate according to claim 7, wherein the step of calculating the recirculation rate takes place using a calculation model defined by the following formula:
$$R = \frac{1 - \frac{{c}_{DO} \cdot {Q}_{D}}{{c}_{sys} \cdot {R}_{D}}}{1 - \frac{{c}_{DO} \cdot {Q}_{D}}{{c}_{sys} \cdot {K}_{D}} + \frac{{c}_{DO} \cdot {Q}_{D}}{{c}_{sys} \cdot {Q}_{B}}}$$
with

| | |
|---|---|
| R | recirculation rate |
| cDO | concentration of a substance at the dialysate output |
| csys | c_sys systemic concentration of a blood component which is |
| | not affected by recirculation (concentration in the patient) |
| QD | dialysis fluid flow rate |
| QB | blood flow rate in the extracorporeal blood line system |
| KD | theoretical clearance of the dialyzer |
| K0A | dialyzer-specific. |
